(19) United States
(12) Patent Application Publication
(10) Pub. No.: US 2004/0030386 A1
(43) Pub. Date: Feb. 12, 2004

(54) Segmentally demineralized bone implant and method for its manufacture

(76) Inventor: Todd Boyce, Aberdeen, NJ (US)

Correspondence Address: Peter G Dilworth, Dilworth & Barnes, 333 Earle Ovington Boulevard, Uniondale, NY, 11553, US

(21) Appl. No.: 10/433,359

(22) PCT Filed: Dec. 3, 2001
(86) PCT No.: PCT/US01/46294

(51) Int. Cl. A61F 2/08; A61F 2/28

(52) U.S. Cl. 623/13.17; 623/23.63; 623/919

(57) ABSTRACT

An improved segmentally demineralized bone implant, useful inter alia as a replacement ligament or tendon possesses at least one demineralized, flexible segment exhibiting reduced osteoinductive properties. The reduction in osteoinductive properties results in the suppression, inhibition or delay of new bone ingrowth in, and consequently remineralizing of, the demineralized segment thereby allowing the segment to retain or prolong its flexible character following insertion of the implant in the body.

### BACKGROUND OF THE INVENTION

1. Field of the Invention

This invention relates to the field of implants, particularly those derived from bone, having use in the repair of ligaments, tendons and for other prosthetic applications.

2. Description of Related Art

U.S. Pat. No. 6,090,998 describes a segmentally demineralized bone implant having at least one mineralized segment and at least one demineralized segment of sufficient flexibility to permit the implant to function as a ligament, tendon or flexible support when the implant is affixed between two or more body parts.

The manufacture of the implant of U.S. Pat. No. 6,090,998 starts with the machining of a segment of preferably cortical bone into a desired shape with at least one end being machined so as to provide a means of fixation of that end, e.g., a thread, directly to a bone machined in a complementary fashion, e.g., a receiving thread. At least one internal segment of the implant is demineralized to a predetermined extent employing known procedures, e.g., acid demineralization, in order to render the segment flexible in the desired degree. This demineralized, flexible segment can be flanked by transition zones that gradually change from demineralized to fully mineralized bone. Optionally, the segmentally demineralized bone implant can be further treated by tanning, e.g., with glutaraldehyde, to reduce its antigenicity.

The implant of U.S. Pat. No. 6,090,998 is indicated to be useful for the repair of ligaments and tendons in the hand, elbow, knee, foot, ankle or other anatomical location and for the replacement of a variety of joints.

While U.S. Pat. No. 6,090,998 is silent regarding the biological properties of the demineralized segment(s) of the implant, it is known that a demineralized bone implant inherently possesses, inter alia, osteoinductive properties which can be expected to manifest themselves when the implant is surgically introduced into the body at a bone repair or other implantation site. Urist et al., “The bone induction principle”, Clin. Orthop., 53:243-83 (1967).

The term “osteoinductive” as used herein shall be understood to refer to the ability of a substance to recruit cells from the host which have osteogenic potential and the ability to form ectopic bone.

The term “osteogenic” as used herein shall be understood to refer to the ability of a substance to induce new bone formation via the participation of living cells from within the substance.

### SUMMARY OF THE INVENTION

This invention provides an improvement in the segmentally demineralized bone implant of U.S. Pat. No. 6,090,998. Specifically, the improvement involves treating at least a portion of the demineralized segment(s) of the bone in order to reduce the osteoinductive properties of the segment(s).

Thus, in accordance with the present invention, in a segmentally demineralized bone implant having at least one mineralized segment and at least one demineralized, flexible segment exhibiting significant osteoinductive properties, the improvement is provided which comprises a demineralized flexible segment at least a portion of which exhibits reduced osteoinductive properties.

The foregoing improved segmentally demineralized bone implant possesses several advantages over the implant described in U.S. Pat. No. 6,090,998, particularly for the replacement of ligaments and tendons as described therein. Thus, reduction of the osteoinductive properties of the demineralized segment(s) of the implant, ranging anywhere from their partial to their substantially complete suppression, inhibition, deactivation or elimination, has the very desirable effect of maintaining the flexibility of the segment(s) for extended periods of time up to and including the entire life of the implant. This, of course, is a valuable characteristic where the implant is required to function as a replacement for a ligament, tendon or other connective tissue, e.g., a meniscus, cartilage, intervertebral disc or stabilization device such as certain cervical plate designs which are intended to allow small amounts of motion so as to accommodate implant subsidence or other changes. In these and similar applications, long-term mobility or flexibility is an important functional requirement of the implant. By contrast, in the implant of U.S. Pat. No. 6,090,998, the untreated demineralized segment(s) retain their full osteoinductive potential and in time, will become remineralized through new bone ingrowth. With remineralization comes reduced flexibility of the originally demineralized segment(s) even to the point where the segment(s) may lack any useful measure of flexibility.

Another significant advantage of the improved segmentally demineralized bone implant of this invention lies in the capacity of certain embodiments of the implant to demonstrate a wider variety of useful biological responses than the implant of U.S. Pat. No. 6,090,998. Thus, unlike the implant of U.S. Pat. No. 6,090,998 whose biological responses are limited to those of its rigid mineralized segment(s), e.g., bone healing and remodeling, and its initially untreated demineralized, flexible segment(s), e.g., osteoinduction accompanied by a loss of flexibility, the improved implant of this invention through suppression of the osteoinductive properties of only a portion of the demineralized segment(s) expands the range of biological responses to include those aforementioned and a third, namely, long-term flexibility in the regions of the demineralized segment(s) whose osteoinductive properties have been curtailed or even eliminated.

### DESCRIPTION OF THE PREFERRED EMBODIMENTS

For the preparation of the segmentally demineralized bone implant of this invention up to and including the demineralization step which is employed to obtain the demineralized, flexible segment(s), one may follow the procedures described in U.S. Pat. No. 6,090,998, the contents of which are incorporated by reference herein. Once demineralization has been effected to provide the desired degree of flexibility in the demineralized segment(s), the implant is ready for further processing in accordance with this invention, specifically, for the step of reducing the osteoinductive properties of at least a portion of the demineralized segment(s). This step can be carried out in a variety of ways, e.g., by contacting the demineralized segments with a chemical denaturation agent to denature or deactivate their osteoinductive proteins, irradiating the demineralized segments with types and levels of irradiation that will also denature or deactivate the osteoinductive proteins, or extracting osteoinductive proteins from the demineralized segments employing suitable extraction agents.

These treatment methods can be utilized individually or in combination and in the case of the latter, can be carried out sequentially or concurrently. The suppression of the osteoinductive properties can be substantially complete or less than complete and can be effected within just a portion of the demineralized segment(s) or the entire region thereof.

In the first of the aforementioned methods, i.e., denaturation of the osteoinductive proteins through the use of a chemical denaturation agent, one or more of these agents are contacted with the demineralized segment(s) of the implant. Useful chemical denaturation agents are those that contain bifunctional or multifunctional reactive groups which react with functional groups on amino acid residues in the osteoinductive proteins, e.g., the epsilon-amine functional group of lysine or hydroxy-lysine or the carboxyl functional groups of aspartic and glutamic acids. Regardless of the precise chemical mechanism that may be involved in the use of a particular chemical denaturation agent, all of the useful chemical denaturation agents herein chemically react with the osteoinductive proteins present in the demineralized segment(s) of the implant to bring about the suppression, diminution, inhibition or substantially complete elimination of their osteoinductive properties.

Suitable chemical denaturation agents include: gases such as ethylene oxide; monoaldehydes and dialdehydes including glutaraldehyde and formaldehyde; polyepoxy compounds such as glycerol polyglycidal ethers, polyethylene glycol diglycidal ethers and other polyepoxy an diepoxy glycidal ethers; tanning agents that include polyvalent metallic oxides such as titanium dioxide, chromium dioxide, aluminum dioxide, zirconium salts, as well as organic tannins and other phenolic oxides derived from plants; chemicals for esterification of carboxyl groups followed by reaction with hydrazide to form activated acyl azide functionalities in the collagen; dicyclohexyl carbodiimide and its derivatives as well as other heterobifunctional denaturation agents; hexamethylene diisocyanate; sugars, including glucose, capable of denaturing proteins such as those present in collagen.

To achieve reaction with osteoinductive proteins in a demineralized segment of the implant, the segment is contacted with the chemical denaturation agent which can be utilized in substantially pure or dilute form, e.g., at levels of from about 0.5 to about 50 weight percent or more, and preferably from about 1 to about 40 weight percent, in a suitable liquid or gaseous diluent for a period known or calculated to provide sufficient reaction with and, consequently, inactivation of, the osteoinductive proteins. Contact times of from about 10 minutes to about 30 days, and preferably from about 4 hours to about 48 hours, are generally effective to achieve this goal. The length of time required will be a function of the activity of the chemical denaturation agent and of the thickness of the demineralized segments. Suitable times for a given denaturation agent can be determined by routine experimentation. The contacting of the demineralized segment(s) with the chemical denaturation agent is conveniently conducted at ambient temperature but can also be accomplished at temperatures above and below ambient, e.g., as low as about 1° C. and as high as about 80° C. Following the foregoing treatment with chemical crosslinking agent, the implant is preferably rinsed free of residual chemical denaturation agent after which the implant can be dried and packaged in any known or conventional manner.

Another useful treatment method for reducing the osteoinductive properties of the demineralized segment(s) of the implant involves subjecting the segmentally demineralized bone to irradiation of sufficient intensity, e.g., gamma irradiation at from about 3-20, and preferably at from about 5-10, Mrads to denature the osteoinductive proteins present in the demineralized bone. Typically, this irradiation treatment will result in substantially complete suppression of the osteoinductive properties of the treated demineralized segment(s) of the implant.

Yet another treatment method suitable for reducing the osteoinductive properties of the demineralized segment(s) of the implant involves the extraction of the osteoinductive proteins present therein. Any of a wide variety of known protein extraction agents can be used for this purpose such as guanidine hydrochloride, high concentrations of salts such as sodium chloride, ammonium sulfate, and the like, urea, etc. Guandine hydrochloride is preferred for this purpose. These and other protein extraction agents can be used for the extraction of the osteoinductive proteins employing procedures that are well known in the art.

Still another method for reducing the osteoinductive properties of the demineralized segment(s) of the implant is to expose the implant to a level and duration of heat that will effect the thermal denaturation of its osteoinductive proteins. Temperatures above about 60° C. and preferably above about 80° C. for periods of time ranging from about 15 minutes to about 16 hours and preferably from about 1 to about 2 hours are generally effective to achieve substantially complete denaturation of the osteoinductive proteins.

In Examples 1-4 which are illustrative of the segmentally demineralized bone implant of the invention and its manufacture, sections of cortical bone which are machined and segmentally demineralized in accordance with procedures described in Examples 1 and 2 of U.S. Pat. No. 6,090,998 are further treated to suppress the osteoinductive activity of the osteoinductive proteins present in their demineralized, flexible segments.

### EXAMPLE 1

The segmentally demineralized implants are placed in an excess of 10% neutral, buffered formalin (a source of formaldehyde) for 48 hours to react with the osteoinductive proteins. Stirring, alternating pressure, sonication, etc., can be used to promote penetration of the formalin solution into the demineralized segments. After treatment with formalin, the implants are rinsed with running water for several hours to remove any residual formalin therefrom.

### EXAMPLE 2

The segmentally demineralized implants are placed in gas-tight containers. After evacuation of the containers, ethylene oxide is introduced therein. The implants are maintained in contact with the ethylene oxide for about 8-12 hours to denature substantially all of the osteoinductive proteins in the demineralized segments. Following evacuation of the ethylene oxide from the containers, the implants are purged with dry nitrogen for from 8to 72 hours to remove any residual ethylene oxide. As a matter of convenience, the foregoing ethylene oxide treatment and subsequent nitrogen purging can be carried out with the implants sealed within terminal sterilization gas-permeable packaging units in accordance with known techniques.

### EXAMPLE 3

The entire segmentally demineralized implants are contacted with 10 ml of 4M guanidine hydrochloride per gram of bone for 8-12 hours at 4° C. to extract osteoinductive proteins from the demineralized segments. After extraction, the implants are rinsed under running water for several hours to remove any residual guanidine hydrochloride.

### EXAMPLE 4

The segmentally demineralized implants are placed in an oven, where they are heated to 80° C. for 36 hours to substantially denature the osteoinductive proteins.

Examples 5-7, below, are further illustrative of the implant of this invention and its manufacture.

### EXAMPLE 5

Specimens are prepared from a human diaphyseal shaft by first making a diaphyseal cut of the appropriate length, followed by longitudinal cuts. In this way, segments are cut to a length of 10 cm. The endosteal and periosteal surfaces of the bone strips are ground on a metallurgical grinding wheel until the cross-sectional dimensions are 1.0+/−0.05 square. The specimen is covered on its end by an elastic, synthetic rubber balloon, fitting tightly around the piece. The specimen is then demineralized using 15 ml of 0.6N HCl per gram of weight for 3 days, until the unmasked region is well-demineralized and flexible.

### EXAMPLE 6

The central region of implant 1 is treated using 4 Mol/L Guanidine HCL with 0.5 Mol/L ethylenediamine tetraacetic acid solution at pH 7.4, using 10 mL solution per gram of bone for 36 hours at 4 degrees C. The solution is changed and the extraction is repeated twice more for 36 hours each. After liberally washed with deionized water and then with 70% ethanol. The sample is packaged and frozen. When implanted between two bony sites, in a ligament application, the implant will be incorporated into the bone at the mineralized ends, and will remain flexible in the demineralized center.

### EXAMPLE 7

The implant of example 1 is treated with 7-9 Mrads of gamma irradiation, after which it is packaged. When implanted between two bony sites, in a ligament application, the implant will be incorporated into the bone at the mineralized ends, and will remain flexible in the demineralized center.

What is claimed is:

1. In a segmentally demineralized bone implant having at least one mineralized segment and at least one demineralized, flexible segment having osteoinductive properties, the improvement which comprises a demineralized segment at least a portion of which exhibits reduced osteoinductive properties.

2. The segmentally demineralized bone implant of claim 1 in which the entire demineralized segment exhibits reduced osteoinductive properties.

3. The segmentally demineralized bone implant of claim 1 wherein the demineralized segment exhibits no appreciable osteoinductive properties.

4. The segmentally demineralized bone implant of claim 1 possessing at least one fully mineralized segment, at least one demineralized segment exhibiting full osteoinductive potential and at least one demineralized segment exhibiting reduced osteoinductive potential.

5. The segmentally demineralized bone implant configured as a ligament or tendon prosthetic device.

6. In a method for manufacturing a segmentally demineralized bone implant in which a segment of bone is subjected to demineralization to render the segment flexible without significantly reducing its osteoinductive properties, the improvement which comprises reducing the osteoinductive properties of at least a portion of the demineralized segment.

7. The method of claim 6 wherein the osteoinductive properties of the entire demineralized segment are reduced.

8. The method of claim 6 wherein the osteoinductive properties of the demineralized segment are reduced to a level where they are substantially completely suppressed, inhibited, deactivated or eliminated.

9. The method of claim 6 wherein reducing the osteoinductive properties of the demineralized segment is carried out by contacting at least a portion of the segment with a chemical denaturation agent which reacts with, and denatures, osteoinductive proteins present in the contacted portion of the segment.

10. The method of claim 6 wherein reducing the osteoinductive properties of the demineralized segment is carried out by exposing at least a portion of the segment to irradiation which denatures osteoinductive proteins present in the exposed portion of the segment.

11. The method of claim 6 wherein reducing the osteoinductive properties of the demineralized segment is carried out by extracting osteoinductive proteins from at least a portion of the demineralized segment employing an osteoinductive protein extraction agent.

12. The method of claim 6 wherein reducing the osteoinductive properties of the demineralized segment is carried out by exposing at least a portion of the demineralized segment to heat at a level and for a time which results in the denaturation of osteoinductive proteins present in the exposed portion of the segment.

13. The method of claim 9 wherein the chemical denaturation agent is at least one member of the group consisting of ethylene oxide, formaldehyde and glutaraldehyde.

14. The method of claim 10 wherein the irradiation is gamma irradiation.

15. The method of claim 11 wherein the osteoinductive protein extraction agent is guanidine hydrochloride.
